(19) United States
(12) Patent Application Publication
(10) Pub. No.: US 2024/0156359 A1
(43) Pub. Date: May 16, 2024

(54) BIO-OPTICAL PHYSIO-LOGGING DEVICE

(71) Applicant: FaunaLabs Inc., Durham, NC (US)

(72) Inventors: David K. Haas, Durham, NC (US); Samuel J. Kelly, Durham, NC (US)

(21) Appl. No.: 18/509,880

(22) Filed: Nov. 15, 2023

Related U.S. Application Data
(60) Provisional application No. 63/383,828, filed on Nov. 15, 2022.

(51) Int. Cl. A61B 5/0205; A61B 5/00; A61B 5/024; A61B 5/08; A61B 5/11; A61B 5/1455

(52) U.S. Cl. CPC A61B 5/0205; A61B 5/0022; A61B 5/0075; A61B 5/02433; A61B 5/0816; A61B 5/11; A61B 5/14552; A61B 5/7271; A61B 5/746; A61B 5/6831; A61B 5/6834; A61B 2560/0443; A61B 2560/0462; A61B 2562/0219; A61B 2562/0223; A61B 2562/0238; A61B 2562/0271; A61B 2562/18

(57) ABSTRACT

A bio-optical physio-logging device designed for attaching to an animal and capturing real-time physiological data of the animal. The bio-optical physio-logging device includes a hardware architecture platform configured to capture physiological signals including the movement, position, and temperature of an animal. The bio-optical physio-logging device is further operable to generate real-time alerts and recommendations based on the animal physiological data.

### CROSS-REFERENCE TO RELATED APPLICATIONS

The present application claims the benefit of U.S. Provisional Patent Application No. 63/383,828 filed on Nov. 15, 2022, the entire content of which is herein incorporated by reference in its entirety.

### BACKGROUND

Physiology research is generally conducted within a controlled laboratory environment due to the inability of recording technologies to monitor wild animals in their natural environment. To truly monitor, analyze, and understand an animal's unique physiological responses, physiology research needs to be performed while an animal is located in their natural environment.

For example, vertebrate animals undergo a constellation of physiological responses when they experience threats from predators or changes in their environmental conditions. These responses might include bradycardia (a reduction in heart rate), tachycardia (an increase in heart rate) and peripheral vasoconstriction (the restriction of oxygenated blood to organs critical to life, for example when exposed to extreme cold) or vasodilation (the opening of blood vessels to provide maximal blood oxygen). This complex cardiovascular set of responses is shown to prioritize the flow of blood oxygen to vital organs, for example supplying blood to the brain during hypoxic events or the muscles in the presence of an immediate threat from a predator. Conversely, the flow of blood oxygen to the brain and heart and the restriction of blood flow to muscle and hypoxic-tolerant tissues is seen during responses to extreme cold. Therefore, in order to successfully monitor and analyze the physiological responses in wild animals, a non-invasive sensor needs to be functional in a plurality of environmental conditions, using attachments that allow the sensing device to stay attached to an animal as it experiences natural life in challenging habitats.

### FIELD OF THE INVENTION

The present invention is generally directed to capturing the physiological data of animals, more specifically to capturing the physiological data of animals using optical sensors.

### DESCRIPTION OF RELATED ART

Real-time physio-logging devices for animals are an unmet need. Current physiological tags cannot simultaneously monitor physiology (e.g., heart rate, peripheral vasoconstriction, and blood oxygenation) and how the physiology varies as a function of an animal's activity state and respiratory cycling. Many real-time physio-logging devices lack sensors capable of measuring and assessing the cardiorespiratory (i.e., heart and lung) health of the animal wearing real-time physio-logging device. Typical physio-logging devices only measure movement (e.g., step count, calorie estimation) or use movement data to infer if an animal is moving (e.g., sitting). Other real-time physio-logging devices are designed to track animal location, however, none of these real-time physio-logging devices can measure cardiopulmonary data (e.g., heart rate, respiratory rate heart rate variability, pulse transit timing, and/or blood oxygen saturation) of animal using bio-optical sensor technologies.

The limitations of current real-time physio-logging devices for capturing real-time physiological data from land animals (i.e., terrestrial vertebrates) are due to the varying nature of the animals'skin, hair, and fur (e.g., thickness, color). Another problem with capturing real-time physiological data is the environment and nature of an animal's movement. For example, when performing physiological response research involving animals, a wearable physiological device needs to be attached in an animal exercise context (e.g., running; escaping a predator). Many studies on the physiological characteristics of animals are performed in a laboratory setting. However, this limits the ability to accurately monitor and analyze the physiological data of an animal because the animal is not being analyzed in its natural environment. Therefore, there is an unmet need for an untethered, non-invasive device capable of measuring the cardiorespiratory health and physiological function of animals.

Yet another unmet need is a non-invasive device designed to monitor companion and agricultural animals with fur and hair (e.g., dogs, cats, cows, sheep). After visiting a veterinarian, many pets need to undergo constant monitoring at home to make sure that medication is being consumed and a post-operation routine is being followed. However, there is currently no solution for providing real-time physiological data of an animal to an owner and/or veterinarian. This results in reliance on the ability of an owner to monitor and care for their pet. The decline in an animal's health is not always visible, especially to an untrained owner, so an owner or a herd manager may wait too long to take their pet to a veterinarian or remove a sick animal from a herd. A non-invasive physiology sensing device would allow pet owners and vets to continuously monitor the treatment and recovery of companion animals and farms to monitor the health and well-being of production animals (e.g., dairy cows). Therefore, there is a need for a non-invasive wearable that monitors the real-time status of animals and provides alerts based on physiological data.

### BRIEF SUMMARY

In one embodiment, the present invention is directed to a bio-optical physio-logging device for monitoring the physiological data of an animal.

In some embodiments, the present invention includes a bio-optical physio-logging device including at least one light emitting component, at least one sensor, a power supply, at least one processor, and at least one antenna. The bio-optical logging device is operable to non-invasively attach to an animal. Once attached to an animal, the at least one processor is operable to activate the at least one light emitting component. The at least one light emitting component is designed to generate a light with a wavelength toward the animal. The at least one sensor is operable to capture a wavelength corresponding to light bouncing off of the animal. The bio-optical physio-logging device is operable to determine at least one physiological condition of the animal based on the captured wavelength.

In some embodiments, the at least one sensor includes at least one of a heart rate sensor, an electrocardiogram (ECG) sensor, an accelerometer, a gyroscope, a magnetometer, a thermometer, a global positioning system component, or a indium-gallium-arsenide photodetector. In some embodiments, the at least one physiological condition includes a pulsative blood value, a oxy hemoglobin spectral absorption value, a deoxy hemoglobin spectral absorption value, a saturation level of peripheral capillary oxygen, an index of tissue perfusion, blood oxygen saturation, or respiratory rate. In some embodiments, based on the pulsative blood value, the at least one processor is configured to determine a time series estimation of a heart rate of the animal. In some embodiments, the at least one sensor is further designed for photoplethysmography and electrocardiography. In some embodiments, the at least one light emitting component is operable to generate a wavelength including a range between about 810 nanometers and about 1300 nanometers. In some embodiments, the at least one sensor includes a solar radiation blocking layer positioned around the at least one sensor. In some embodiments, the bio-optical physio-logging device further includes an attachment component that includes at least one of a strap, a suction cup, a hook and loop mechanism, and/or a snap connector.

In some embodiments, the present invention includes a bio-optical physio-logging device including at least one light emitting component, at least one optical sensor including a layer of radiation blocking material, a power supply, at least one processor, and at least one antenna. The layer of radiation blocking material is positioned around an exterior of the at least one optical sensor. The bio-optical physio-logging device is operable to non-invasively attach to an animal. The bio-optical physio-logging device is in network communication with at least one remote device. Once attached to the animal, the at least one processor is operable to activate the at least one light emitting component. The at least one light emitting component is designed to generate a light with a wavelength toward the animal. The at least one optical sensor is operable to capture a wavelength corresponding to the light bouncing off of the animal. Based on the captured wavelength, the bio-optical physio-logging device is operable to determine at least one physiological condition of the animal. Based on the at least one determined physiological condition of the at least one processor generate at least one alert. The at least one alert is transmitted to the at least one remote device.

In some embodiments, the at least one optical sensor includes a photodetector and is designed for photoplethysmography and/or near-infrared spatially-resolved diffuse reflectance spectroscopy (SRDS) bio-optical sensing. The at least one optical sensor is operable to detect variation in reflected light or transmitted light. In some embodiments, the at least one light emitting component is operable to generate a multi-wavelength light. The at least one optical sensor is further operable to receive a reflected wavelength for each wavelength of the multi-wavelength light. The at least one processor is operable to determine at least one physiological condition for each reflected wavelength. In some embodiments, the bio-optical physio-logging device further includes a position monitoring sensor operable to monitor at least one of a direction, speed, and/or movement of the animal. The at least one processor is further operable to determine at least one physiological condition based on the at least one direction, speed, and/or movement of the animal. In some embodiments, the at least one physiological condition includes a pulsative blood value, a oxy hemoglobin spectral absorption value, a deoxy hemoglobin spectral absorption value, a saturation level of peripheral capillary oxygen, an index of tissue perfusion, blood oxygen saturation, and/or a respiratory rate of the animal. In some embodiments, the wavelength of the light generated by the at least one light emitting component is between about 800 nanometers and about 1300 nanometers.

In some embodiments, the present invention includes a bio-optical physio-logging device including at least one optical sensor, at least one position sensor, at least one light emitting diode, at least one processor, and at least one antenna. The bio-optical physio-logging device is operable to attach to at least one animal. Once attached to the at least one animal, the bio-optical physio-logging device is in contact with skin of the at least one animal. The at least one light emitting diode is configured to generate light with at least two diodes. Each wavelength of the at least two wavelengths is different. The at least one optical sensor includes a photodetector. The at least one optical sensor is designed for photoplethysmography and/or near-infrared spatially-resolved diffuse reflectance spectroscopy (SRDS) bio-optical sensing. The at least one optical sensor is operable to detect variation in reflected light or transmitted light. The at least one position sensor is operable to detect and monitor movement of the at least one animal. The at least one processor is configured to activate the at least one light emitting diode. The generated light travels to within the skin of the at leas one animal. The at least one optical sensor is operable to receive reflected light from the at least one animal. Based on the receive reflected light, the at least one processor is operable to determine at least one physiological condition of the at least one animal.

In some embodiments, the at least one animal includes a terrestrial animal or a marine animal. In some embodiments, the at least one processor is operable to determine variations in arterial blood based on the variation in the reflected light. In some embodiment, the at least one optical sensor includes a layer of radiation blocking material. The radiation blocking material wraps around the at least one optical sensor. In some embodiments, the at least one physiological condition includes a pulsative blood value, a oxy hemoglobin spectral absorption value, a deoxy hemoglobin spectral absorption value, a saturation level of peripheral capillary oxygen, an index of tissue perfusion, blood oxygen saturation, or respiratory rate.

In another embodiment, the present invention is directed to a bio-optical physio-logging device, including a hardware architecture platform configured to capture physiological signals, including the movement, position, and temperature of an animal.

In yet another embodiment, the present invention includes a bio-optical physio-logging device designed to provide real-time alerts and analysis based on the physiological data of an animal.

### BRIEF DESCRIPTION OF THE SEVERAL VIEWS OF THE DRAWINGS

The embodiments illustrated, described, and discussed herein are illustrative of the present invention. As these embodiments of the present invention are described with reference to illustrations, various modifications or adaptations of the methods and or specific structures described may become apparent to those skilled in the art. It will be appreciated that modifications and variations are covered by the above teachings and within the scope of the appended claims without departing from the spirit and intended scope thereof. All such modifications, adaptations, or variations that rely upon the teachings of the present invention, and through which these teachings have advanced the art, are considered to be within the spirit and scope of the present invention. Hence, these descriptions and drawings should not be considered in a limiting sense, as it is understood that the present invention is in no way limited to only the embodiments illustrated.

FIG. 1 illustrates a bio-optical physio-logging device system according to one embodiment of the present invention.

FIG. 2A illustrates a side view of a bio-optical physio-logging device according to one embodiment of the present invention.

FIG. 2B illustrates a bottom view of a bio-optical physio-logging device according to one embodiment of the present invention.

FIG. 3A illustrates a bottom perspective of an exploded view of a bio-optical physio-logging device according to one embodiment of the present invention.

FIG. 3B illustrates a top perspective of an exploded view of a bio-optical physio-logging device according to one embodiment of the present invention.

FIG. 4 illustrates a bottom perspective view of a bio-optical physio-logging device according to one embodiment of the present invention.

FIG. 5A illustrates a side view of a bio-optical physio-logging device according to one embodiment of the present invention.

FIG. 5B illustrates a front view of a bio-optical physio-logging device according to one embodiment of the present invention.

FIG. 5C illustrates a bottom view of a bio-optical physio-logging device according to one embodiment of the present invention.

FIG. 6 illustrates a schematic diagram of a sensor component of a bio-optical physio-logging device according to one embodiment of the present invention.

FIG. 7A illustrates a dashboard of a software platform according to one embodiment of the present invention.

FIG. 7B illustrates a dashboard of a software platform according to one embodiment of the present invention.

FIG. 7C illustrates a dashboard of a software platform according to one embodiment of the present invention.

FIG. 7D illustrates a dashboard of a software platform according to one embodiment of the present invention.

FIG. 7E illustrates a dashboard of a software platform according to one embodiment of the present invention.

FIG. 7F illustrates a dashboard of a software platform according to one embodiment of the present invention.

FIG. 7G illustrates a dashboard of a software platform according to one embodiment of the present invention.

FIG. 8A illustrates physiological data of a cow from a bio-optical physio-logging device according to one embodiment of the present invention.

FIG. 8B illustrates optical data of a cow from a bio-optical physio-logging device according to one embodiment of the present invention.

FIG. 8C illustrates movement data of a cow from a bio-optical physio-logging device according to one embodiment of the present invention.

FIG. 9 illustrates a schematic diagram of a bio-optical physio-logging system according to one embodiment of the present invention.

FIG. 10 illustrates a schematic diagram of server of a bio-optical physio-logging system according to one embodiment of the present invention.

FIG. 11 illustrates a schematic diagram of a computer of a bio-optical physio-logging system according to one embodiment of the present invention.

FIG. 12 illustrates a schematic diagram of mobile device of a bio-optical physio-logging system according to one embodiment of the present invention.

FIG. 13 illustrates a schematic diagram of an IoT device of a bio-optical physio-logging system according to one embodiment of the present invention.

### DETAILED DESCRIPTION

For the purposes of promoting an understanding of the present disclosure, reference will be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “a composite” means at least one composite and can include more than one composite.

Throughout the specification, the terms “about” and/or “approximately” may be used in conjunction with numerical values and/or ranges. The term “about” is understood to mean those values near to a recited value. For example, “about 40 [units]”may mean within +/−25% of 40 (e.g., from 30 to 50), within +/−20%, +/−15%, +/−10%, +/−9%, +/−8%, +/−7%, +/−6%, +/−5%, +/−4%, +/−3%, +/−2%, +/−1%, less than +/−1%, or any other value or range of values therein or there below. Furthermore, the phrases “less than about [a value]” or “greater than about [a value]” should be understood in view of the definition of the term “about” provided herein. The terms “about” and “approximately” may be used interchangeably.

As used herein, the verb “comprise” as is used in this description and in the claims and its conjugations are used in its non-limiting sense to mean that items following the word are included, but items not specifically mentioned are not excluded.

Throughout the specification the word “comprising,” or variations such as “comprises” or “comprising,” will be understood to imply the inclusion of a stated element, integer or step, or group of elements, integers, or steps, but not the exclusion of any other element, integer or step, or group of elements, integers or steps. The present disclosure may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only”, and the like in connection with the recitation of claim elements, or the use of a “negative” limitation.

Unless defined otherwise, all technical and scientific terms used herein have the same meanings as commonly understood by one of ordinary skill in the art to which this disclosure belongs. Preferred methods, devices, and materials are described, although any methods and materials similar or equivalent to those described herein can be used in the practice or testing of the present disclosure. All references cited herein are incorporated by reference in their entirety.

Photoplethysmography (PPG) works through illumination of epidermal and dermal tissue. Variation in pulsatile, oxygen-saturated arterial blood in the dermis is detected as variation in either received reflected light (reflectance pulse oximetry) or received transmitted light (transmittance pulse oximetry). The received light is collected at a photodetector, where a current is generated. The current is sampled by an analog-to-digital converter and converted to a numeric value indicating the intensity of light delivered to the photodetector. In the absence of ambient light, motion artifacts, electrical interference, and poor perfusion of illuminated tissues, the digitized variation in pulsatile blood volume is an effective time series for estimation of heart rate. When multiple wavelengths of light are used to illuminate tissues, and those two wavelengths have significant variation in their oxy- and deoxy-hemoglobin spectral absorption values, the ratio of each wavelengths' received intensity from pulsatile arterial blood flow (or alternating current) divided by its received intensity of venous and non-pulsatile arterial blood and other chromophore absorption (or direct current), can be used to compute a continuous estimate of blood oxygen saturation.

In one embodiment, the present invention includes an Internet-of-Things (IoT) device and a hardware architecture platform designed for data collection and real-time streaming of vertebrate physiological, movement, and position data. The present invention is further configured to measure physiological signals (e.g., heart rate, blood oxygen saturation, and tissue perfusion) using bio-optical sensors and/or electrocardiogram sensors, movement (i.e., pitch, roll, and heading) using inertial measurement units, position (e.g., latitude, longitude, and altitude via GPS), temperature, and altitude. For example, and not limitation, the present invention is designed for real-time collection and streaming of sensor data, operable to receive software updates via network communication, and attach to an animal for physiological data capture. Advantageously, the present invention includes a contact sensor column designed to interface with a body surface of a tagged animal that is designed for near-infrared spatially resolved diffuse reflectance spectroscopy (SRDS) bio-optical sensing, and movement tracking using an inertial movement unit. SRDS employs one or more multi-wavelength light source(s) and/or one or more photodetectors (PD), spaced at increasing source-detector separation distances, for the characterization of reflectance differences over a greater range of tissue depths. As a further example, and not limitation, the present invention is configured to determine animal heart rate and detect cardiac arrhythmia. The present invention is further operable to determine respiratory rate using optical sensing.

In one embodiment, the present invention includes a software platform designed to measure physiological signals (e.g., heart rate, blood oxygen saturation, and tissue perfusion using bio-optical and electrocardiogram sensors), animal movement (e.g., walking, eating, drinking, licking, biting) via inertial measurement units, position (i.e., latitude, longitude, and altitude via a Global Positioning system (GPS)), temperature, altitude, and/or pressure. For example, and not limitation, in one embodiment, heart rate is determined by measuring the variation resulting from the pulsatile component of arterial blood flow. For example, and not limitation, in one embodiment, the software platform is a web application platform. The software platform is designed to receive data from bio-optical physio-logging devices and attachable devices including physiological, movement, and ecological data. In one embodiment, the software platform is designed for terrestrial animals. The software platform further includes a storage component. For example, and not limitation, the software platform is designed to stream high resolution data to secondary storage devices (e.g., microSD cards) and stream real-time sensor data over common internet protocols (e.g., a web browser via HTTP; MQTT).

In another embodiment, the software platform is designed to work with microcontrollers designed for wired, Wi-Fi, Bluetooth, cellular/LTE, and most sub-1 GHz wireless standards. For example, and not limitation, in one embodiment, the processors include digital signal processors. Advantageously, the present invention is designed for the collection, wireless streaming, and/or real-time analysis of multi-sensor data inputs (e.g., wearable devices for large and small animals, wearable human heart rate and blood oxygenation monitors, smart-home thermostats, chemical sensors, lighting, security).

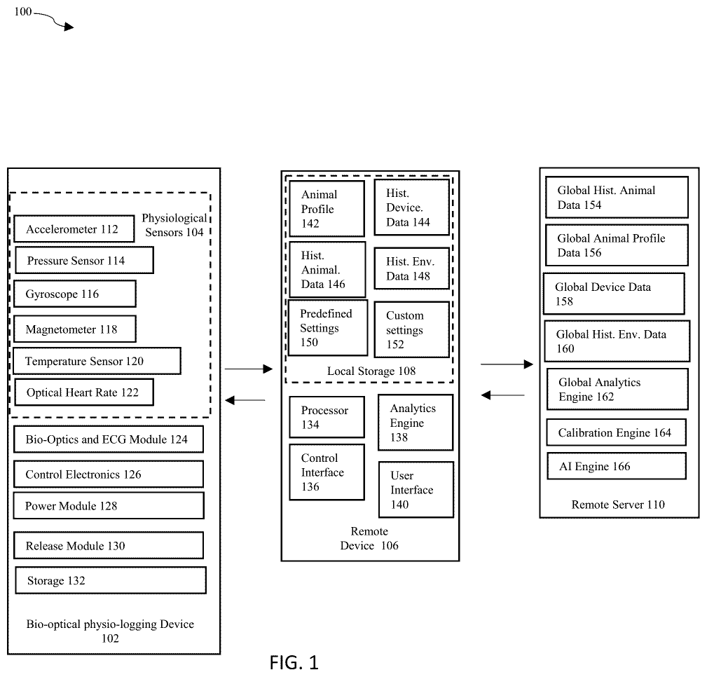

FIG. 1 illustrates a block diagram of a bio-optical physio-logging system according to one embodiment of the present invention. The bio-optical physio-logging device system includes a bio-optical physio-logging device 102 including a plurality of physiological sensors 104, a remote device 106 with local storage 108, and a remote server 110. The plurality of physiological sensors includes, but is not limited to, an accelerometer 112, a pressure sensor 114, a gyroscope 116, a magnetometer 118, a temperature sensor 120, and an optical heart rate sensor 122.

The bio-optical physio-logging device 102 is designed to attach to an animal and capture physiological data corresponding to the animal. For example, and not limitation, the physiological data includes heart rate, blood oxygen saturation, respiratory rate, and index of tissue perfusion. The animal includes, but is not limited to, vertebrate animals inhabiting terrestrial environments. The present invention further includes at least one inertial measurement unit (IMU). For example, and not limitation, the IMU includes, but is not limited to, at least one accelerometer, at least one gyroscope, and/or at least one magnetometer. For example, and not limitation, in one embodiment, at least one IMU includes an MPU-9250, an ICM-20948, and/or a Bosch BN0055. Advantageously, the IMU is designed to determine kinematic patterns (e.g., walking, running, eating, drinking, for bio-optical noise cancellation) of a subject. The system further includes a temperature sensor and a pressure sensor. For example, and not limitation, the present invention includes a Bosch BME-280, and/or Keller PA-series or the TE Connectivity MS5837-30BA pressure sensors.

For example, and not limitation, the present invention includes an analog-front-end integrated circuit (i.e., Texas Instruments AFE 4900 analog-front-end integrated circuit). The integrated circuit is designed to support synchronized photoplethysmography (PPG) and electrocardiography (ECG) of human and non-human subjects. Advantageously, the bio-optical physio-logging device is designed to work with a software platform for optical bio-sensing applications. For example, and not limitation, the software platform includes algorithms designed for heart-rate monitoring (HRM) and saturation of peripheral capillary oxygen (SPO2). The PPG signal is in electrical communication with a plurality of diodes. For example, and not limitation, the plurality of diodes includes at least four switching light-emitting diodes (LEDs) and at least one photodiode (PD) (e.g., 3 photodiodes) designed for spatial-resolved diffuse reflectance spectroscopy (SRDRS). Advantageously, the LEDs are designed to generate a light including a number corresponding to the PPG signal. The hardware module (AFE4900) includes a LED driver, a trans-impedance amplifier (TIA) to convert currents from up to three photodetectors, and an analog-digital converter (ADC) for digitization of the photodetector's photon counts.

For further example, and not limitation, light from the LEDs enters the biological tissues of the animal under observation. Light at the specific wavelengths used in the bio-optical physio-logging device, which may range between about 900 to about 1300 nm, is absorbed to varying extents by the blood underlying the skin of the animal under observation. A percentage of that light is reflected back through the skin, hair and/or fur of the animal under observation. The remaining reflected light is collected at the photodiode(s). As the amount of blood oxygen varies with each heartbeat and breathing cycle, the amount of each wavelength of light returning to the photodiode(s) is used to measure heart rate, blood oxygen saturation, and respiration rate.

The accelerometer 112 is configured to track movement in a plurality of directions. The accelerometer is operable to receive inertial measurements (e.g., velocity and positioning) of an animal. The accelerometer is designed to sense inclination, tilt, and orientation of the animal's body. Additionally, the accelerometer is operable to detect forward and backward movement and a rate of speed change. This data is used to infer the observed animal's behavior (e.g., eating, drinking, licking, scratching). The accelerometer data is used by the device software, by the remote server and/or the remote device to describe the physical activity, the physical activity intensity, and the total energy expenditure. When attached in a way that brings the device into contact with the chest of the observed animal, the accelerometer is used to measure animal heart rate using seismocardiography (the measurement of heart rate using accelerometry data) and to improve the integrity of the bio-optical physiology signal through noise-cancelation methods.

The gyroscope 116 measures the orientation and rotation of an animal. In one embodiment, the present invention includes a 3-axis gyroscope and a 3-axis accelerometer to provide a 6-degree of freedom motion tracking system. When attached in a way that brings the device into contact with the chest of the observed animal, the gyroscope is used to measure animal heart rate using gyrocardiography (the measurement of heart rate using gyroscope data). The magnetometer 118 is designed to measure magnetic fields (e.g., direction, strength, and/or relative change) at a particular location.

The temperature sensor 120 monitors an animal's body temperature and tracks temperature changes. In one embodiment, the temperature sensor includes a thermometer. For example, and not limitation, the noninvasive body temperature sensor includes an infrared temperature sensor.

The heart rate sensor 122 is operable to capture the heart rate, respiratory rate, and heart rate variability of an animal. For example, and not limitation, the heart rate is determined using photoplethysmography, electrocardiography, pulse oximetry, ballistocardiography, seismocardiography, and/or gyrocardiography. For example, and not limitation, in one embodiment, the heart rate sensor is designed to measure heart (precordial) vibrations via contact with an animal. In yet another embodiment, the heart rate sensor relies on accelerometer data to measure precordial linear acceleration and the gyroscope to measure precordial angular velocity. The heart rate data enables the present invention to determine an animal's intensity while moving and/or undergoing unique physical movements (e.g., diving). In one embodiment, the heart rate sensor is incorporated into the bio-optical physio-logging device. For example, and not limitation, in one embodiment, the heart rate sensor includes an optical sensor designed to use light on the skin to measure a pulse of an animal. In some embodiments, the heart rate sensor is a combination of a bio-optical sensor and an accelerometer and/or gyroscope.

In one embodiment, the Bio-Optics and ECG Module 124 is designed to receive the data captured by the physiological sensors and perform optical physiology analysis and ECG measurement using the physiological data. In another embodiment, the control electronics 126 include an electromagnetic interference shield, an electrocardiogram (ECG) circuit, a global positioning system (GPS) antenna, a radio-frequency (RF) receiver and/or transmitter for sending and receiving information using WiFi, cellular, LoRa, and/or satellite telemetry systems, a microcontroller, a plurality of light emitting diodes (LEDs), a plurality of photodetectors (PDs), an inductive current drive configured to drive an inductive coil, a lithium polymer (LiPo) charging subsystem comprising a LiPo battery connector, a charging circuit, a switch bypass, a battery fuel gauge, a power supply, and a load switch, a flash memory, a microcontroller reset control package, a power sensing control module, a sensor sleep component, and a debug connector.

In yet another embodiment, the control electronics includes a voltage sensing circuit, an analog-to-digital converter (ADC), a processor, the indicator, and optionally a driver. The voltage sensing circuit can be any standard voltage sensing circuit, such as those found in volt meters. An input voltage VIN is supplied via the power BUS. In one embodiment, the voltage sensing circuit includes standard amplification or de-amplification functions for generating an analog voltage that correlates to the amplitude of the input voltage VIN that is present. The ADC receives the analog voltage from the voltage sensing circuit and performs a standard analog-to-digital conversion.

The processor manages the overall operations of the bio-optical physio-logging device. The processor is any controller, microcontroller, or microprocessor that is capable of processing program instructions. In one embodiment, the control electronics includes at least one antenna, which enables the bio-optical physio-logging device to send information (e.g., location, physiological data) to at least one remote device (e.g., smartphone, tablet, laptop computer) and/or receive information (e.g., timing commands, power commands) from at least one remote device. At least one antenna provides wireless communication, standards-based or non-standards-based, by way of example and not limitation, radiofrequency (RF), WI-FI, BLUETOOTH, ZIGBEE, NEAR FIELD COMMUNICATION (NFC), LORA, LORAWAN, 3G, 4G, and/or 5G CELLULAR, SATELLITE TELEMETRY SYSTEMS or other similar communication methods.

The present invention further includes a hardware architecture designed to work with a plurality of microcontrollers and digital signal processor families. For example, and not limitation, the present invention is designed to work with a CC3220R, CC3230SF, and CC3235SF families of secure microcontrollers and network processors. The present invention is configured to work with the MSP432 microcontroller family designed for Wi-Fi capability provided by the CC312 AND CC3130 network processors. The present invention is further designed for CC26XX and CC13xx family of microcontrollers for applications incorporating Bluetooth and sub-1 GHz wireless protocols. The present invention is further designed to work with any ARM Cortex-M4 microcontroller.

In one embodiment, the power module 128 includes a rechargeable lithium-ion or lithium-polymer battery (e.g., 3.7 volts) and a plurality of battery cells (e.g., 150 mAh capacity or 2500 mAh capacity). The present invention further includes a Qi-compatible inductive charging receiver designed to charge the lithium battery. Advantageously, the present invention further includes a software platform designed to support a plurality of charging rates. For example, and not limitation, in one embodiment, the plurality of charging rates includes a charging rate of about 100 milliamperes, about 500 milliamperes, and greater than about 1 ampere. The present invention further includes an integrated circuit designed to protect against under and over-charging. Additionally, the integrated circuit is designed to provide battery power output to a second integrated circuit (e.g., 60 nanoampere quiescent current (IQ), 1.8 volts to 6.5 volts input voltage, high-efficiency 750-mA step-down converter (i.e., TPS62840), a low-dropout output power supply with selectable regulated power ranges between about 1.98 volts to about 3.3 volts). The system further includes a load switch to provide regulated power to the bio-optical physio-logging device (i.e., MCU and/or DSP, sensors, telemetry tracking circuits).

The physio-logging device includes a frequency-selectable beacon. For example, and not limitation, the frequency-selectable beacon includes an amplitude modulation (AM), frequency modulation (FM), and/or a very high frequency (VHF) beacon. The physio-logging device further includes an integrated circuit (e.g., Silicon Labs SI514) as an RF carrier wave generator. Advantageously, RF tracking enables the physio-logging device to be detected, tracked, and recovered in outdoor environments via the RF signals. In one embodiment, the physio-logging device is configured to work between 50 MHz and 230 MHz.

The bio-optical physio-logging device 102 with physiological sensors 104, the remote device 106 with local storage 108, and the remote server 110 are designed to connect directly (e.g., Universal Serial Bus (USB) or equivalent) or wirelessly (e.g., Bluetooth®, Wi-Fi®, ZigBee®) through systems designed to exchange data between various data collection sources. In a preferred embodiment, bio-optical physio-logging device 102 with the physiological sensor 104, the remote device 106 with local storage 108, and the remote server 110 communicate wirelessly through Wi-Fi.

The local storage 108 on the remote device 106 includes an animal profile 142, historical device data 144, historical animal data 146, historical environmental data 148, predefined animal settings 150, and predefined custom settings 152. The animal profile data 142 includes data corresponding to at least one target animal (age, weight, size, height, animal type, diet, etc.). The historical device data 144 includes data captured by the bio-optical physio-logging device and status information of the physiological sensors 104, the Bio-Optics and ECG module 124, control electronics 126, power module 128, and release module 130. The historical device data includes data from the accelerometer 112, the pressure sensor 114, the gyroscope 116, the magnetometer 118, the temperature sensor 120, and the optical heart rate sensor 122. The historical animal data 146 includes historical physiological data of at least one target animal using the bio-optical physio-logging device. For example, and not limitation, the historical physiological data includes heart rate, respiratory rate, blood pressure, movement, temperature, and other animal physiological data. The historical environment data 148 includes data relating to at least one target animal's environment. For example, and not limitation, the historical environment data includes habitat type (e.g., marine, desert, forest, grassland, savannah, subterranean, etc.), temperature, humidity, oxygen levels, wind, soil composition, elevation, predators, parasites, and precipitation.

The remote server includes global historical animal data 154, global animal profile data 156, global device data 158, global historical environmental data 160, a global analytics engine 162, a calibration engine 164, and an artificial intelligence engine 166. The global target animal data 154, the global animal profile data 156, the global device data 158, the global environment data 160, the global analytics engine 162, the calibration engine 164, and the artificial intelligence engine 166 includes animal data for plurality of target animals, a plurality of animal species, and/or a plurality of environments.

In one embodiment, the present invention further includes an artificial intelligence engine 166 operable to receive real-time or near real-time data from the bio-optical physio-logging device 102 and the remote device 106. For example, and not limitation, in one embodiment, the artificial intelligence engine includes at least one machine learning algorithm. The artificial intelligence engine includes a visualization component. The visualization component is operable to display the captured and analyzed real-time or near real-time data. The visualization tool is operable to generate and display bar graphs, line graphs, circle graphs, histograms, a flow chart, a control chart, a scatter plot, anatomical diagrams, and other graphical displays to illustrate the captured data.

In yet another embodiment, the artificial intelligence engine is operable to generate a real-time or near real-time alert based on the real-time or near real-time physiological data. The at least one alert includes changes in environmental conditions (ex. temperature increase), changes in animal data (e.g., heart rate) changes in bio-optical physio-logging device activity (e.g., sensor or power malfunctioning), and other similar alerts relating to the monitoring of an animal. Additionally, the present invention is configured to provide a recommendation based on the alert. The present invention is further operable to automatically contact a third party based on the physiological data. For example, and not limitation, the present invention is operable to detect that the power and/or efficiency of the bio-optical physio-logging device is declining and to request maintenance on the bio-optical physio-logging device.

In one embodiment, the present invention is designed to monitor a plurality of animals. For example, and not limitation, in one embodiment, the present invention is designed to monitor a herd of cows. The present invention is designed to monitor the physiological data and generate real-time alerts and recommendations based on the physiological data. For example, and not limitation, the present invention is configured to generate an alert based on a cow's estrus, stress, and sickness. The present invention is further operable to monitor supplemental feeding, grazing location, milking frequency, and disease outbreak. Advantageously, this enables the real-time monitoring and disease detection of cows, thereby improving the care of the herd of cows.

In yet another embodiment, the present invention is operable to generate real-time alerts based on at least one animal's heart rate, breathing rate, stress level, sleep, activity (e.g., scratching, eating, step count), and/or location. Advantageously, the present invention is configured to monitor for diseases (e.g., cardiovascular disease (e.g., hyperthyroidism, heart murmur)) based on the real-time physiological data. The present invention is further configured to transmit the real-time data and alerts to third-party. For example, and not limitation, the present invention is configured to transmit an alert to a veterinarian device based on the real-time physiological data. The veterinarian device is operable to receive at least one treatment recommendation via a user interface and transmit the at least one treatment recommendation to the present invention. Advantageously, this enables real-time updates and recommendations to and from a veterinarian device. The present invention is further configured to determine the efficacy of outpatient treatment based on the real-time physiological data, alerts, and recommendations.

In one embodiment, the remote server includes a software platform. The software platform includes an online mode, an armed mode, a high-resolution data sampling and archival data collection mode, an analysis mode, and a hibernation mode. In another embodiment, the online mode enables the software platform to be accessed via a web browser. The present invention further includes a web interface and a web application server designed for lower-power microcontrollers. The software platform is designed to receive input to configure the bio-optical physio-logging wearable device hardware and software. The present invention is configured for real-time sensor data streaming (e.g., real-time heart rate and saturated blood oxygen monitoring; movement data) via binary data format over wire (USB or serial cables) and via MOTT over one or more wireless interfaces (e.g., via a web browser), or customized network-capable application using TCP/IP or UDP IP packets.). The present invention is further designed to perform over-the-wire or over-the-air downloads of high-resolution archival data, prepare the bio-optical physio-logging device for remote time and/or conditional deployments or uses (e.g., activate high-resolution sensor recording at a specific altitude or any other sensor condition), automatic release, sleep mode or long-term low-power hibernation based on specific conditions, initiate over-the-air (OTA) software updates of tag OS when the device is connected to the public internet, monitor the status and health of the sensor of the bio-optical physio-logging device and the power subsystem and act as its own Wi-Fi network (AP mode) or join an existing Wi-Fi mode (STA mode).

While in armed mode, the bio-optical physio-logging device is designed to operate in a lower-power mode while evaluating sensor data at a low frequency (e.g., about 5 Hz) for conditions that may trigger higher-power high-resolution data sampling and archival data collection.

While in the high resolution data sampling and archival data collection mode, the software platform is configured to perform optimized, deterministic real-time sampling of sensor data, with periodic recording of the high-resolution sample data to a secondary storage device (e.g., microSD card). In one embodiment, the data is compressed using a data compression algorithm. The high high-resolution archival data is offloaded using the bio-optical physio-logging device in online mode using a wired (e.g., USB cable) or over-the air (over HTTP). Once the archival data is downloaded, the application is decompressed and post-processes the binary data format for analysis. The application uses binary file format and plain text (comma and tab separated).

While in analysis mode, the software platform is configured for real-time analysis of the physiological data. The real-time analysis includes processing of raw bio-optical sensor and/or ECG sensor data to calculate instantaneous heart rate, average heart rate, estimate saturated blood oxygenation, index of tissue of perfusion, and experimental support for computing heart rate variability and respiration rate, pitch, roll, and heating, depth or altitude (terrestrial context). The analytics techniques include machine learning (ML), fast Fourier transform (FFT), and wavelet analysis methods. Hibernation mode includes optimizing the bio-optical physio-logging device hardware and software for the lowest possible ultra-low power deep sleep or suspend state.

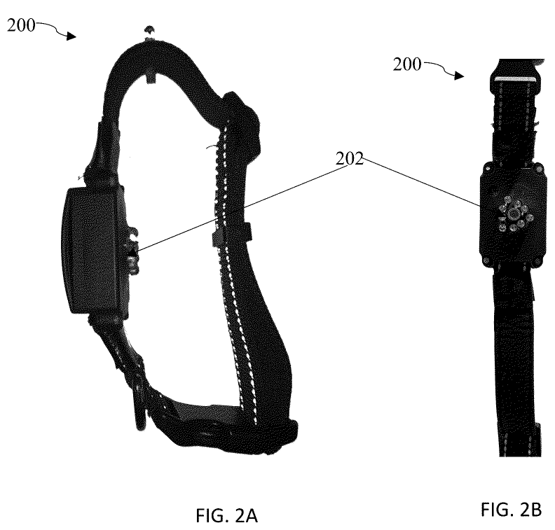

FIG. 2A illustrates a side perspective of a bio-optical physio-logging device according to one embodiment of the present invention. FIG. 2B illustrates a bottom perspective of a bio-optical physio-logging device according to one embodiment of the present invention. In one embodiment, the bio-optical physio-logging device 200 is designed to attach to an animal via at least one attachment component. For example, and not limitation, the attachment component includes a hook and loop mechanism, a snap connector, a strap, a suction cup, and other similar attachment mechanisms. Advantageously, the bio-optical physio-logging device is designed such that at least one sensor 202 is positioned against an animal's skin.

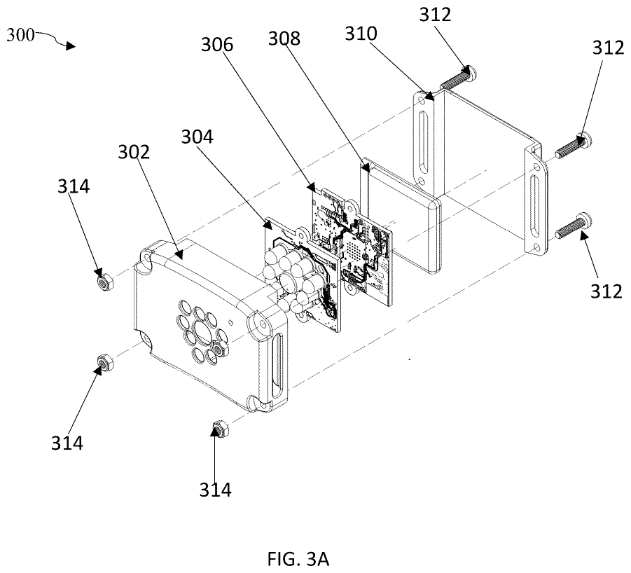

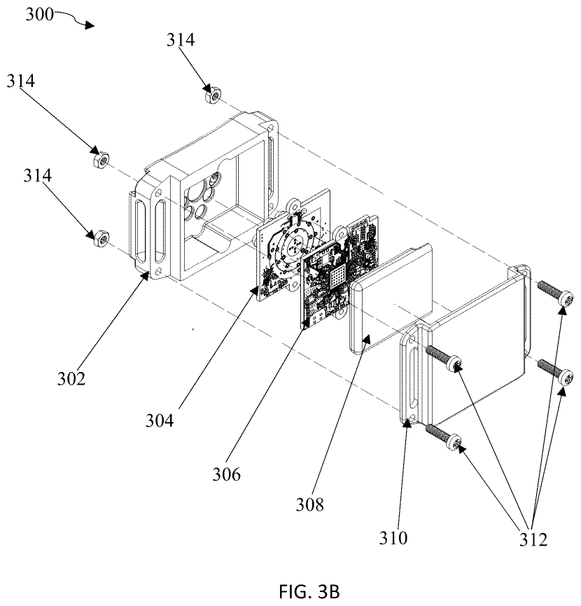

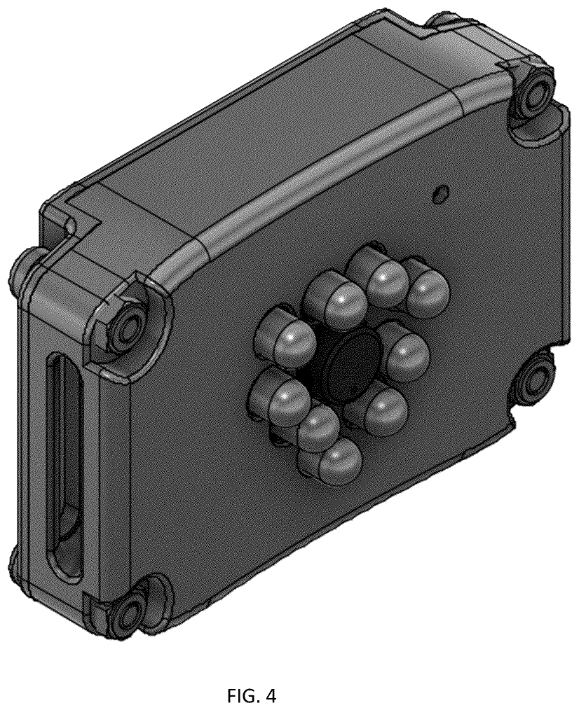

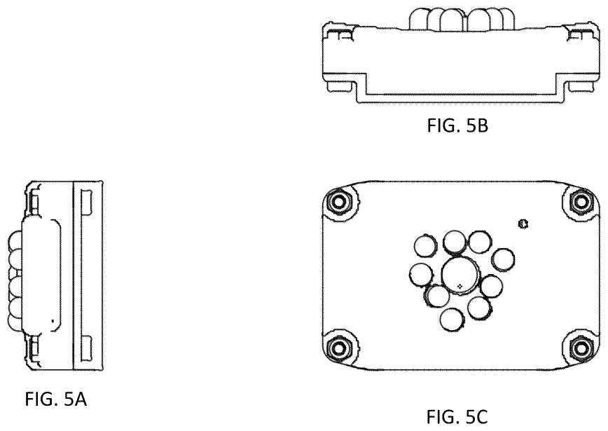

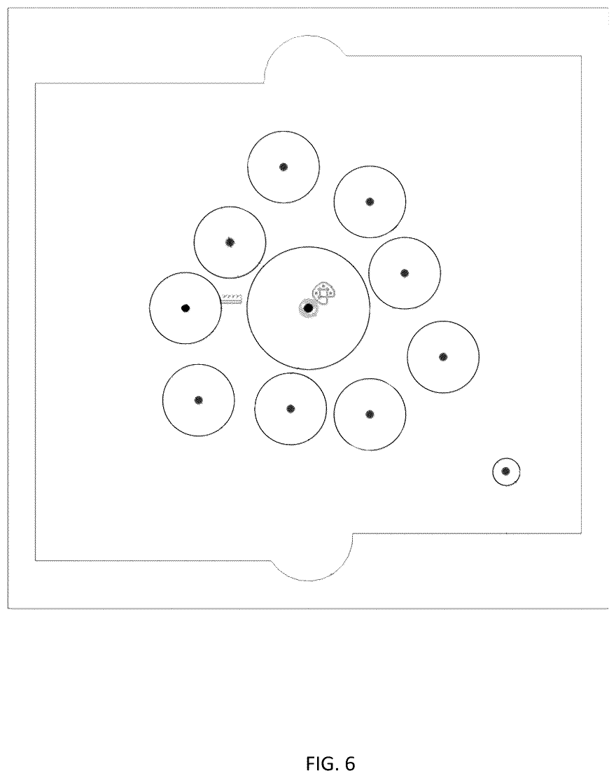

FIG. 3A illustrates a bottom perspective view of a bio-optical physio-logging device according to one embodiment of the present invention. FIG. 3B illustrates a top perspective view of a bio-optical physio-logging device according to one embodiment of the present invention. The bio-optical physio-logging device 300 includes a bottom collar case 302, a sensor printed circuit board assembly 304, a printed circuit board assembly 306, a battery 308, and a top case 310. In some embodiments, the top collar case 310 is operable to receive an attachment component 312 (e.g., screw, bolt) and the bottom case 302 is operable to receive a holding component 314 (e.g., nut) to receive the attachment component. FIG. 4 illustrates a bottom perspective of a bio-optical physio-logging device according to one embodiment of the present invention. FIG. 5A illustrates a side view of a bio-optical physio-logging device according to one embodiment of the present invention. FIG. 5B illustrates a front view of a bio-optical physio-logging device according to one embodiment of the present invention. FIG. 5C illustrates a front view of a bio-optical physio-logging device according to one embodiment of the present invention

FIG. 6 illustrates a schematic diagram of a sensor component of a bio-optical physio-logging device according to one embodiment of the present invention. In some embodiments, the bio-optical physio-logging device includes a plurality of light emitting diodes. In some embodiments, each light emitting diode of the plurality of light emitting diodes have a diameter of about 5.25 millimeters. In some embodiments at least one diode has a diameter of about 9.00 millimeters. In some embodiments, a center of a central diode is about 7.50 millimeters away from a center of another diode. In some embodiments, at least one diode is positioned at a forty degree angle relative to a center of a central diode. For further example, and not limitation, the sensor includes a plurality of diodes including at least four switching light-emitting diodes (LEDs) and at least one photodiode (PD) (e.g., 3 photodiodes) designed for spatial-resolved diffuse reflectance spectroscopy (SRDRS). Advantageously, the LEDs are designed to generate a light including a number corresponding to the PPG signal. The bio-optical physio-logging device includes a hardware module including a LED driver, a trans-impedance amplifier (TIA) to convert currents from up to three photodetectors, and an analog-digital converter (ADC) for digitization of the photodetector's photon counts.

For further example, and not limitation, light from the LEDs enters the biological tissues of the animal under observation. Light at the specific wavelengths used in the bio-optical physio-logging device, which may range between about 900 to about 1300 nm, is absorbed to varying extents by the blood underlying the skin of the animal under observation. A percentage of that light is reflected back through the skin, hair and/or fur of the animal under observation. The remaining reflected light is collected at the photodiode(s). As the amount of blood oxygen varies with each heartbeat and breathing cycle, the amount of each wavelength of light returning to the photodiode(s) is used to measure heart rate, blood oxygen saturation, and respiration rate.

In one embodiment, this bio-optical sensor component employs three independent near-infrared wavelengths of light, ranging between 810 nanometers and 1300 nanometers. In one embodiment, these light-emitting sources are near-infrared light-emitting diodes (LEDs). Light emitting sources of a given wavelength are spaced at a constant distance from one or more indium-gallium-arsenide (InGaAs) photodetectors (PDs) to maximize the collection of reflected light containing physiology information from the observed animal. In one embodiment, an InGaAs photodetector is located centrally with respect to three sets of three near-infrared light-emitting diodes operating with peak light emission between 940 nanometers and 980 nanometers, 1050 nanometers, and 1200 nanometers, and, respectively, at distances from the central InGaAs photodetector of 7.5 millimeters, 9.5 millimeters, and 10.5 millimeters.

Ambient near-Infrared Radiation (NIR) from the sun adversely affects the bio-optical physio-logging device's optical sensor and negatively affects data capture. To address this problem, a layer of metallic (e.g., gold, silver) foil was wrapped around the optical sensor, which prevents 99% of ambient solar radiation from entering the optical sensor. Advantageously, this protects the optical sensor from solar radiation and improves the accuracy of data collection. Additionally, the sensor includes a layer of metallic foil on the top inside section of the sensor housing, which further prevents intrusion of solar radiation. In yet another embodiment, the device enclosure is manufactured with an inclusion of black pigmentation, which further absorbs ambient solar radiation, resulting in an almost complete minimization of the negative effect of solar radiation.

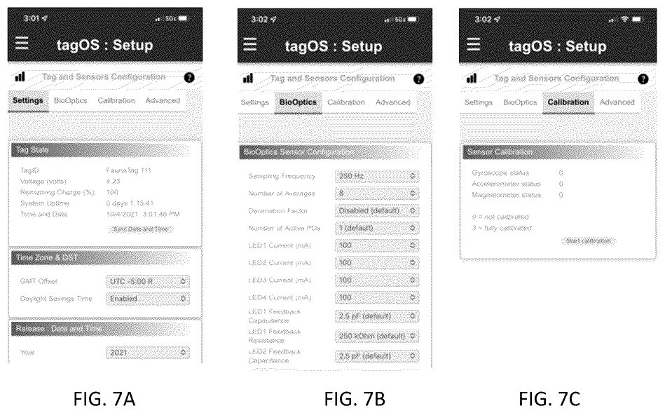

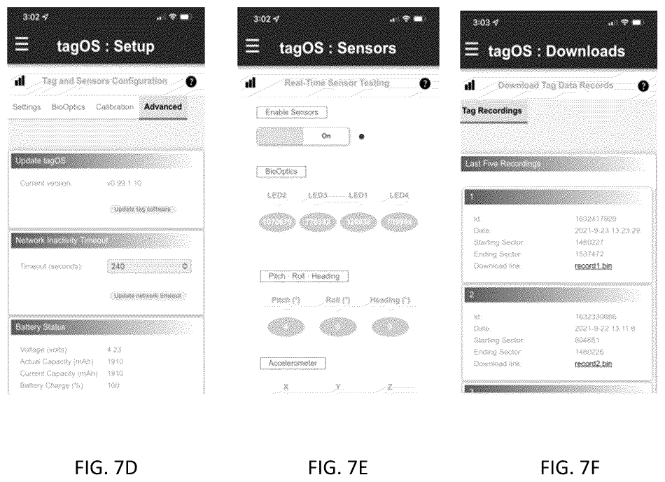

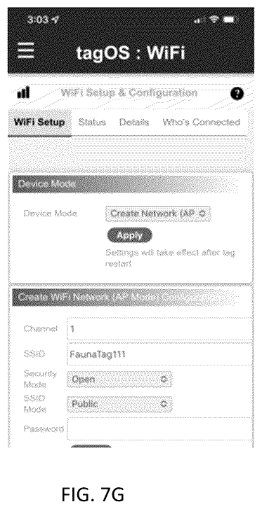

FIGS. 7A-7G illustrate a plurality of dashboards of a software platform according to one embodiment of the present invention. FIG. 7A illustrates the state of a bio-optical physio-logging device (e.g., voltage, charge, uptime, time, and date). FIG. 7B illustrates a sensor configuration according to one embodiment of the present invention. For example, and not limitation, the sensor configuration includes sampling frequency, number of averages, decimation factor, LED currents, LED Feedback Capacitance, and LED Feedback Resistance. FIG. 7C illustrates a sensor calibration according to one embodiment of the present invention. For example, and not limitation, the present invention is operable to display and monitor the calibration status of the gyroscope, accelerometer, and magnetometer.

As shown in FIG. 7D, the present invention is further operable to display the current software version of the bio-optical physio-logging device and the battery status (e.g., voltage, actual capacity, current capacity, and battery charge). As shown in FIG. 7E, the software platform is further operable to send commands to enable the sensors of the bio-optical physio-logging device, provide the real-time status of each LED of the bio-optical physio-logging device, the pitch, roll, and heading of the IMU, and the movement in the X, Y, and Z direction based on accelerometer data. FIG. 7F illustrates the time and location where a recording was made using the bio-optical physio-logging device. As shown in FIG. 7G, the present invention is further configured to create a local Wi-Fi network.

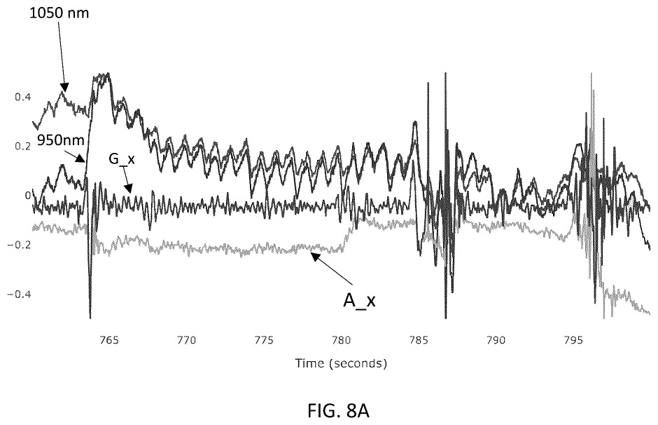

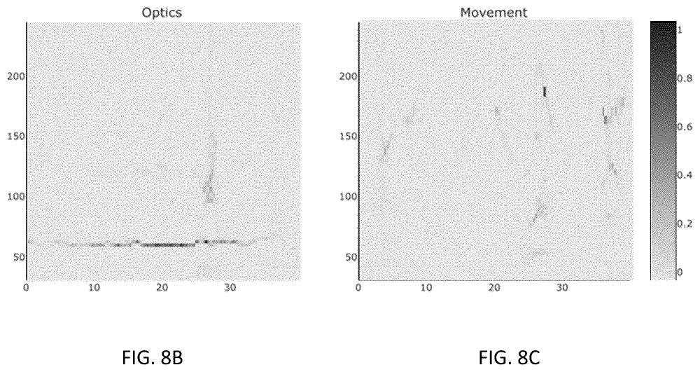

FIG. 8A illustrates a graph of physiological data corresponding to a cow using a bio-optical physio-logging device according to one embodiment of the present invention. FIG. 8A is representative raw data of the optically measured heart rate signal without signal processing. The first and second data channels represent active near-infrared optical channels and show a steady periodic change consistent with heartbeat. A third optical channel, which measures ambient light, shows no indication of heartbeat, strengthening the claim that the first and second optical channels are measuring cardiopulmonary physiology. A fourth and fifth data channel, which are measurements of movement using accelerometers (A_x) and gyroscopes (G_x), also show no indication of heartbeat, which further strengthen the claim for optical measurement of cardiopulmonary data using data from non-invasive near-infrared optics on the first and second data channel. FIG. 8B illustrates a graph that includes optics of a cow using a bio-optical physio-logging device according to one embodiment of the present invention. This frequency domain analysis of the first and second optical data channels is used to generate the heart rate profile of the cow under observation, revealing a steady heart rate of about 60 heartbeats per minute. FIG. 8C illustrates a movement graph of a cow using the physio-logging device according to one embodiment of the present invention. This graph employs the same frequency domain analysis used with the first and second active bio-optical physiology channels in FIG. 8B, but reveals no sign of heartbeat, which strengthens the claim that near-infrared bio-optics are measuring physiology and that this measurement is not a movement artifact.

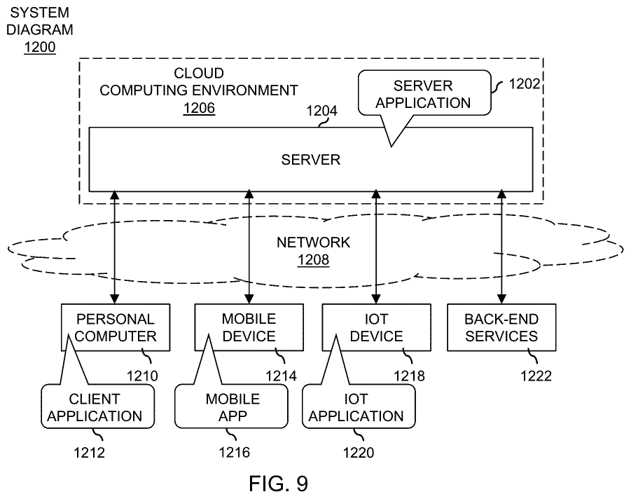

FIG. 9 depicts a system diagram 1200 illustrating a client/server architecture in accordance with embodiments of the present disclosure. The server application 1202 is configured to provide a video application and mobile application for a smart multi-resident community management system. A server application 1202 is hosted on a remote server 1204 within a cloud computing environment 1206. The server application 1202 is provided on a non-transitory computer-readable medium including a plurality of machine-readable instructions, which when executed by one or more processors of the server 1204, are adapted to cause the server 1204 to generate the video platform and mobile application.

The server application 1202 is configured to communicate over a network 1208. In a preferred embodiment, the network 1208 is the Internet. In other embodiments, the network 1208 may be restricted to a private local area network (LAN) and/or private wide area network (WAN). The network 1208 provides connectivity with a plurality of client devices including a personal computer 1210 hosting a client application 1212, a mobile device 1214 hosting a mobile app 1216. The network 1208 also provides connectivity for an Internet-Of-Things (IoT) device 1218 hosting an IoT application 1220 and to back-end services 1222. Advantageously, the back-end services are operable to communicate with third-party application programming interfaces (APIs) to either provide or receive data that can be used by the system to provide recommendations. Third-party applications provide algorithms for analysis of data. The back-end services may provide data gathered within the bio-optical physio-logging systems through the third-party APIs and receives results from the algorithms provided back to the back-end services to provide further recommendations or take further actions within the bio-optical physio-logging system.

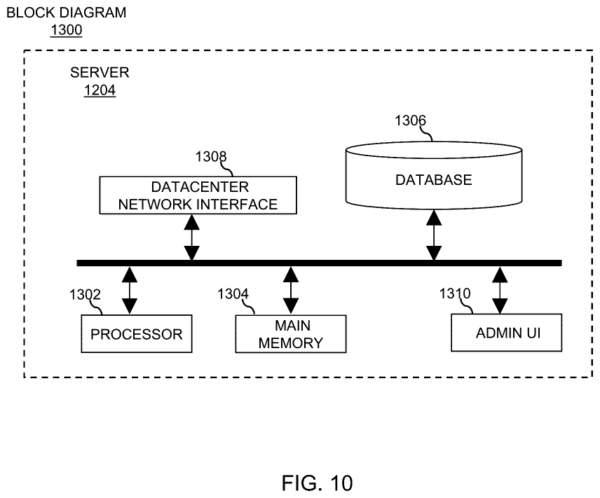

FIG. 10 depicts a block diagram 1300 of the server 1204 of FIG. 9 for hosting at least a portion of the server application 1202 of FIG. 9 in accordance with embodiments of the present disclosure. The server 1204 may be any of the hardware servers referenced in this disclosure. The server 1204 may include at least one of a processor 1302, a main memory 1304, a database 1306, a datacenter network interface 1308, and an administration user interface (UI) 1310. The server 1204 may be configured to host one or more virtualized servers. For example, the virtual server may be an Ubuntu® server or the like. The server 1204 may also be configured to host a virtual container. For example, the virtual server may be the DOCKER® virtual server or the like. In some embodiments, the virtual server and or virtual container may be distributed over a plurality of hardware servers using hypervisor technology.

The processor 1302 may be a multi-core server class processor suitable for hardware virtualization. The processor 1302 may support at least a 64-bit architecture and a single instruction multiple data (SIMD) instruction set. The memory 1304 may include a combination of volatile memory (e.g., random access memory) and non-volatile memory (e.g., flash memory). The database 1306 may include one or more hard drives.

The datacenter network interface 1308 may provide one or more high-speed communication ports to the data center switches, routers, and/or network storage appliances. The datacenter network interface may include high-speed optical Ethernet, InfiniB and (IB), Internet Small Computer System Interface iSCSI, and/or Fibre Channel interfaces. The administration UI may support local and/or remote configuration of the server by a data center administrator.

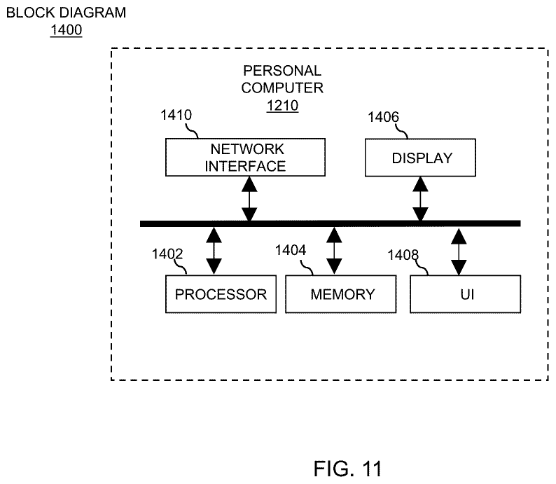

FIG. 11 depicts a block diagram 1400 of the personal computer 1210 of FIG. 9 in accordance with embodiments of the present disclosure. The personal computer 1210 may be any of the devices referenced in this disclosure. The personal computer 1210 may include at least a processor 1402, a memory 1404, a display 1406, a user interface (UI) 1408, and a network interface 1410. The personal computer 1210 may include an operating system to run a web browser and/or the client application 1212 shown in FIG. 9. The operating system (OS) may be a Windows® OS, a Macintosh® OS, or a Linux® OS. The memory 1404 may include a combination of volatile memory (e.g., random access memory) and non-volatile memory (e.g., solid state drive and/or hard drives).

The network interface 1410 may be a wired Ethernet interface or a Wi-Fi interface. The personal computer 1210 may be configured to access remote memory (e.g., network storage and/or cloud storage) via the network interface 1410. The UI 1408 may include a keyboard, and a pointing device (e.g., mouse). The display 1406 may be an external display (e.g., computer monitor) or internal display (e.g., laptop). In some embodiments, the personal computer 1210 may be a smart TV. In other embodiments, the display 406 may include a holographic projector.

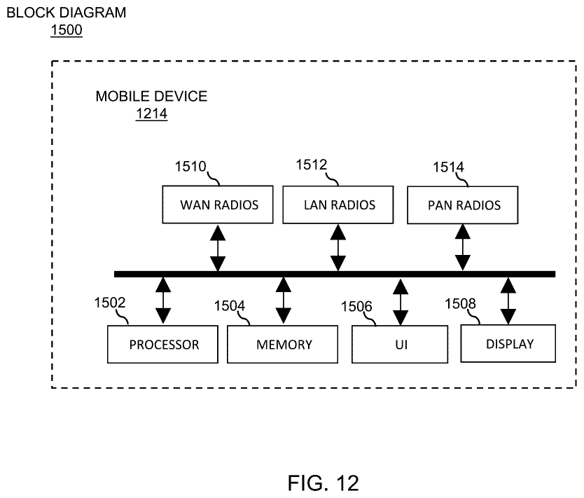

FIG. 12 depicts a block diagram 1500 of the mobile device 1214 of FIG. 9 in accordance with embodiments of the present disclosure. The mobile device 1214 may be any of the remote devices referenced in this disclosure. The mobile device 1214 may include an operating system to run a web browser and/or the mobile app 1216 shown in FIG. 9. The mobile device 1214 may include at least a processor 1502, a memory 1504, a UI 1506, a display 1508, WAN radios 1510, LAN radios 1512, and personal area network (PAN) radios 1514. In some embodiments the mobile device 1214 may be an iPhone® or an iPad®, using iOS® as an OS. In other embodiments the mobile device 1214 may be a mobile terminal including Android® OS, BlackBerry® OS, Chrome® OS, Windows Phone® OS, or the like.

In some embodiments, the processor 1502 may be a mobile processor such as the Qualcomm® SnapdragonTM mobile processor. The memory 1504 may include a combination of volatile memory (e.g., random access memory) and non-volatile memory (e.g., flash memory). The memory 1504 may be partially integrated with the processor 1502. The UI 1506 and display 1508 may be integrated such as a touchpad display. The WAN radios 1510 may include 2G, 3G, 4G, and/or 5G technologies. The LAN radios 1512 may include Wi-Fi technologies such as 802.11a, 802.11b/g/n, and/or 802.11ac circuitry. The PAN radios 1514 may include Bluetooth® technologies.

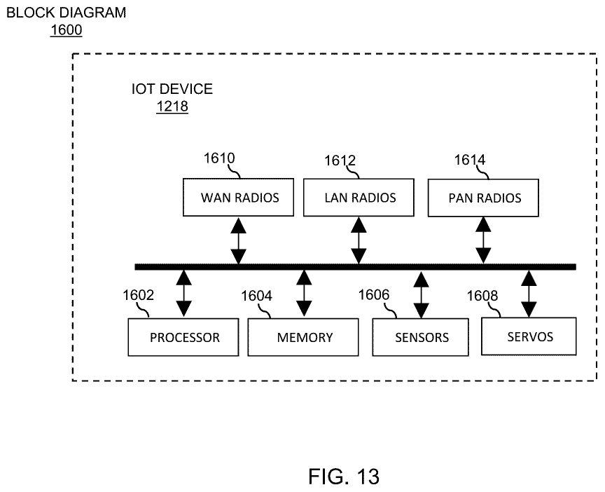

FIG. 13 depicts a block diagram 1600 of the IoT device 1218 of FIG. 9 in accordance with embodiments of the present disclosure. The IoT device 1218 may be any of the remote devices referenced in this disclosure. The IoT device 1218 includes a processor 1602, a memory 1604, sensors 1606, servos 1608, WAN radios 1610, LAN radios 1612, and PAN radios 1614. The processor 1602, a memory 1604, WAN radios 1610, LAN radios 1612, and PAN radios 1614 may be of similar design to the processor 1502, a memory 1504, WAN radios 1510, LAN radios 1512, and PAN radios 1514 of the mobile device 1214 of FIG. 9. The sensors 1606 and servos 1608 may include any applicable components related to IoT devices such as a monitoring device, an autonomous vehicle, a home assistant, a smart appliance, a medical device, a virtual reality device, an augmented reality device, or the like.

Any combination of one or more computer-readable medium(s) may be utilized. The computer-readable medium may be a computer readable signal medium or a computer-readable storage medium (including, but not limited to, non-transitory computer-readable storage media). A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer readable storage medium and that can communicate, propagate, or transport a program for use by or in connection with an instruction execution system, apparatus, or device.

In one embodiment, the present invention includes a cloud-based network for distributed communication via a wireless communication antenna and processing by at least one mobile communication computing device. In another embodiment of the invention, the system is a virtualized computing system capable of executing any or all aspects of software and/or application components presented herein on computing devices. In certain aspects, the computer system may be implemented using hardware or a combination of software and hardware, either in a dedicated computing device, or integrated into another entity, or distributed across multiple entities or computing devices.

By way of example, and not limitation, the computing devices are intended to represent various forms of digital computers and mobile devices, such as a server, blade server, mainframe, mobile phone, personal digital assistant (PDA), smartphone, desktop computer, netbook computer, tablet computer, workstation, laptop, and other similar computing devices. The components shown here, their connections and relationships, and their functions, are meant to be exemplary only, and are not meant to limit implementations of the invention described and/or claimed in this document.

In one embodiment, the computing device includes components such as a processor, a system memory having a random-access memory (RAM) and a read-only memory (ROM), an I2C sensor, and a system bus that couples the memory to the processor. In another embodiment, the computing device may additionally include components such as a storage device for storing the operating system and one or more application programs, a network interface unit, and/or an input/output controller. Each of the components may be coupled to each other through at least one bus. The input/output controller may receive and process input from, or provide output to, a number of other devices, including, but not limited to, alphanumeric input devices, mice, electronic styluses, display units, touch screens, signal generation devices (e.g., speakers), or printers.

By way of example, and not limitation, the processor may be a general-purpose microprocessor (e.g., a central processing unit (CPU)), a graphics processing unit (GPU), a microcontroller, a Digital Signal Processor (DSP), an Application Specific Integrated Circuit (ASIC), a Field Programmable Gate Array (FPGA), a Programmable Logic Device (PLD), a controller, a state machine, gated or transistor logic, discrete hardware components, or any other suitable entity or combinations thereof that can perform calculations, process instructions for execution, and/or other manipulations of information.

In another embodiment, multiple processors and/or multiple buses may be used, as appropriate, along with multiple memories of multiple types (e.g., a combination of a DSP and a microprocessor, a plurality of microprocessors, one or more microprocessors in conjunction with a DSP core).

Also, multiple computing devices may be connected, with each device providing portions of the necessary operations (e.g., a server bank, a group of blade servers, or a multi-processor system). Alternatively, some steps or methods may be performed by circuitry that is specific to a given function. According to various embodiments, the computer system may operate in a networked environment using logical connections to local and/or remote computing devices through a network. A computing device may connect to a network through a network interface unit connected to a bus. Computing devices may communicate communication media through wired networks, direct-wired connections or wirelessly, such as acoustic, RF, or infrared, through an antenna in communication with the network antenna and the network interface unit, which may include digital signal processing circuitry when necessary. The network interface unit may provide for communications under various modes or protocols.

In one or more exemplary aspects, the instructions may be implemented in hardware, software, firmware, or any combinations thereof. A computer readable medium may provide volatile or non-volatile storage for one or more sets of instructions, such as operating systems, data structures, program modules, applications, or other data embodying any one or more of the methodologies or functions described herein. The computer readable medium may include the memory, the processor, and/or the storage media and may be a single medium or multiple media (e.g., a centralized or distributed computer system) that store the one or more sets of instructions. Non-transitory computer readable media includes all computer readable media, with the sole exception being a transitory, propagating signal per se. The instructions may further be transmitted or received over the network via the network interface unit as communication media, which may include a modulated data signal such as a carrier wave or other transport mechanism and includes any delivery media. The term “modulated data signal” means a signal that has one or more of its characteristics changed or set in a manner as to encode information in the signal.

Storage devices and memory include, but are not limited to, volatile and non-volatile media such as cache, RAM, ROM, EPROM, EEPROM, FLASH memory, or other solid state memory technology; discs (e.g., digital versatile discs (DVD), HD-DVD, BLU-RAY, compact disc (CD), or CD-ROM) or other optical storage; magnetic cassettes, magnetic tape, magnetic disk storage, floppy disks, or other magnetic storage devices; or any other medium that can be used to store the computer readable instructions and which can be accessed by the computer system.

The various illustrative logical blocks, modules, elements, circuits, and algorithms described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application (e.g., arranged in a different order or partitioned in a different way), but such implementation decisions should not be interpreted as causing a departure from the scope of the present invention.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of code, which comprises one or more executable instructions for implementing the specified logical function(s). It should also be noted, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A bio-optical physio-logging device comprising:
at least one light emitting component;
at least one sensor;
a power supply;
at least one processor; and
at least one antenna;
wherein the bio-optical physio-logging device is operable to non-invasively attach to an animal;
wherein, once attached to the animal; the at least one processor is operable to activate the least one light emitting component;
wherein the at least one light emitting component is designed to generate a light with a wavelength toward the animal;
wherein the at least one sensor is operable to capture a wavelength corresponding to light bouncing off of the animal; and
wherein, based on the captured wavelength, the bio-optical physio-logging device is operable to determine at least one physiological condition of the animal.

2. The device of claim 1, wherein the at least one sensor includes at least one of a heart rate sensor, an electrocardiogram (ECG) sensor, an accelerometer, a gyroscope, a magnetometer, a thermometer, a global positioning system component, or a indium-gallium-arsenide photodetector.

3. The device of claim 2, wherein the at least one physiological condition includes a pulsative blood value, a oxy hemoglobin spectral absorption value, a deoxy hemoglobin spectral absorption value, a saturation level of peripheral capillary oxygen, an index of tissue perfusion, blood oxygen saturation, or respiratory rate.

4. The device of claim 3, wherein, based on the pulsative blood value, the at least one processor is configured to determine a time series estimation of a heart rate of the animal.

5. The device of claim 3, wherein the at least one sensor is further designed for photoplethysmography and electrocardiography.

6. The device of claim 1, wherein the at least one light emitting component is operable to generate a wavelength including a range between about 810 nanometers and about 1300 nanometers.

7. The device of claim 2, wherein the at least one sensor includes a solar radiation blocking layer, wherein the solar radiation blocking layer is positioned around the at least one sensor.

8. The device of claim 1, further including an attachment component, wherein the attachment component includes at least one of a strap, a suction cup, a hook and loop mechanism, and/or a snap connector.

9. A bio-optical physio-logging device comprising:
at least one light emitting component;
at least one optical sensor including a layer of radiation blocking material;
a power supply;
at least one processor; and
at least one antenna;
wherein the layer of radiation blocking material is positioned around an exterior of the at least one optical sensor;
wherein the bio-optical physio-logging device is operable to non-invasively attach to an animal;
wherein the bio-optical physio-logging device is in network communication with at least one remote device;
wherein, once attached to the animal; the at least one processor is operable to activate the least one light emitting component;
wherein the at least one light emitting component is designed to generate a light with a wavelength toward the animal;
wherein the at least one optical sensor is operable to capture a wavelength corresponding to light bouncing off of the animal;
wherein, based on the captured wavelength, the bio-optical physio-logging device is operable to determine at least one physiological condition of the animal; and
wherein based on the at least one determined physiological condition, the at least one processor generates at least one alert, wherein the at least one alert is transmitted to the at least one remote device.

10. The device of claim 9, wherein the at least one optical sensor includes a photodetector, wherein the at least one optical sensor is designed for photoplethysmography and/or near-infrared spatially-resolved diffuse reflectance spectroscopy (SRDS) bio-optical sensing, wherein the at least one optical sensor is operable to detect variation in reflected light or transmitted light.

11. The device of claim 9, wherein the at least one light emitting component is operable to generate a multi-wavelength light, wherein the at least one optical sensor is operable to receive a reflected wavelength for each wavelength of the multi-wavelength light, wherein the at least one processor is operable to determine at least one physiological condition for each reflected wavelength.

12. The device of claim 11 further comprising a position monitoring sensor, wherein the position monitoring sensor is operable to monitor at least one of a direction, speed, and/or movement of the animal, wherein the at least one processor is further operable to determine at least one physiological condition based on the at least one direction, speed, and/or movement of the animal.

13. The device of claim 9, wherein the at least one physiological condition includes a pulsative blood value, a oxy hemoglobin spectral absorption value, a deoxy hemoglobin spectral absorption value, a saturation level of peripheral capillary oxygen, an index of tissue perfusion, blood oxygen saturation, and/or a respiratory rate of the animal.

14. The device of claim 9, wherein the wavelength of the light generated by the at least one light emitting component is between about 800 nanometers and about 1300 nanometers.

15. A bio-optical physio-logging device comprising:
at least one optical sensor;
at least one position sensor;
at least one light emitting diode;
at least one processor; and
at least one antenna;
wherein the bio-optical physio-logging device is operable to attach to at least one animal, wherein, when attached to the at least one animal, the bio-optical physio-logging device is in contact with skin of the at least one animal;
wherein the least one light emitting diode is configured to generate light with at least two wavelengths, wherein each wavelength of the at least two wavelength is different;
wherein the at least one optical sensor includes a photodetector, wherein the at least one optical sensor is designed for photoplethysmography and/or near-infrared spatially-resolved diffuse reflectance spectroscopy (SRDS) bio-optical sensing, wherein the at least one optical sensor is operable to detect variation in reflected light or transmitted light;
wherein the at least one position sensor is operable to detect and monitor movement of the at least one animal;
wherein the at least one processor is configured to activate the at least one light emitting diode, wherein the generated light travels to within the skin of the at least one animal, wherein the at least one optical sensor is operable to receive reflected light from the at least one animal; and
wherein, based on the received reflected light, the at least one processor is operable to determine at least one physiological condition of the at least one animal.

16. The device of claim 15, wherein the at least one animal includes a terrestrial animal or a marine animal.

17. The device of claim 15, wherein the at least one processor is operable to determine variations in arterial blood based on the variation in the reflected light.

18. The device of claim 15, wherein the at least one optical sensor includes a layer of radiation blocking material, wherein the radiation blocking material wraps around the at least one optical sensor.

19. The device of claim 18, wherein the radiation blocking material includes gold or silver.

20. The device of claim 15, wherein the at least one physiological condition includes a pulsative blood value, a oxy hemoglobin spectral absorption value, a deoxy hemoglobin spectral absorption value, a saturation level of peripheral capillary oxygen, an index of tissue perfusion, blood oxygen saturation, or respiratory rate.
